(19) United States
(12) Patent Application Publication
(10) Pub. No.: US 2014/0185416 A1
(43) Pub. Date: Jul. 3, 2014

(54) MENSTRUAL CYCLE MONITOR

(71) Applicant: Alison Elizabeth Sanchez, Mississauga (CA)

(72) Inventor: Alison Elizabeth Sanchez, Mississauga (CA)

(21) Appl. No.: 14/123,554

(22) PCT Filed: Jul. 6, 2012
(86) PCT No.: PCT/CA2012/000657

§ 371 Date: Dec. 3, 2013

(30) Foreign Application Priority Data
Jul. 7, 2011 (GB) GB-1111651.4

(51) Int. Cl. A61B 10/00

(52) U.S. Cl. CPC A61B 10/0012
USPC 368/10

(57) ABSTRACT

The monitor comprises beads threaded onto a loop of elastic cord. The cord is formed into a loop by tying the two ends of a length of cord in a reef-knot. The daily progress of the menstrual cycle is marked by pulling the knot through holes in the beads. The beads are coloured to indicate the sectors of the cycle—red for menstruation, pink for fertility, black for PMS. The knot is matched to the elastic cord and to the hole in the bead so that the knot is tight in the hole, but the pull-through force is smaller than the elastic limit of the cord.

This technology relates to a device for increasing awareness and knowledge of the menstrual cycle. As such, the device is intended to be used mainly by pubescent girls.

The device can be used by women for monitoring the sectors of the menstrual cycle, including indicating premenstrual sysndrome and the sector of the cycle in which fertility—i.e the likelihood or probability of conception—is enhanced. It is not intended that the device be used for contraceptive purposes.

### LIST OF DRAWINGS

FIG. 1 is a plan view of a bracelet of beads, which embodies the described technology. The beads are of different colours, as described.

FIG. 2 is a pictorial view of a bead of the bracelet.

FIG. 3 is a plan view of the bracelet, which differs from FIG. 1 in showing the bracelet during movement of a knot through one of the beads. FIG. 3 shows how a person applies the forces needed to pull the knot through the bead.

FIG. 4 is a plan view of the knot used to secure the cord on which the beads of the bracelet have been threaded.

FIG. 5 is a sectioned side-view of one of the beads, and shows the knot being pulled through a through-hole of the bead.

FIG. 6 is an end view from the right of FIG. 4, and shows the profile of the knot as it lies inside the through-hole.

FIG. 7 is the same view as FIG. 6, but shows the knot outside the bead.

FIG. 8 is the same view as FIG. 1, and depicts an inscribed circle of the bracelet.

The scope of the patent protection sought herein is defined by the accompanying claims. The apparatuses and procedures shown in the accompanying drawings and described herein are examples.

FIGS. 1,3 depict a bracelet 30, in which a set 32 of twenty-eight beads have been threaded onto a cord 34. The cord, with the beads threaded thereon, has been formed into an endless loop, and the ends of the cord have been tied together by a knot 36.

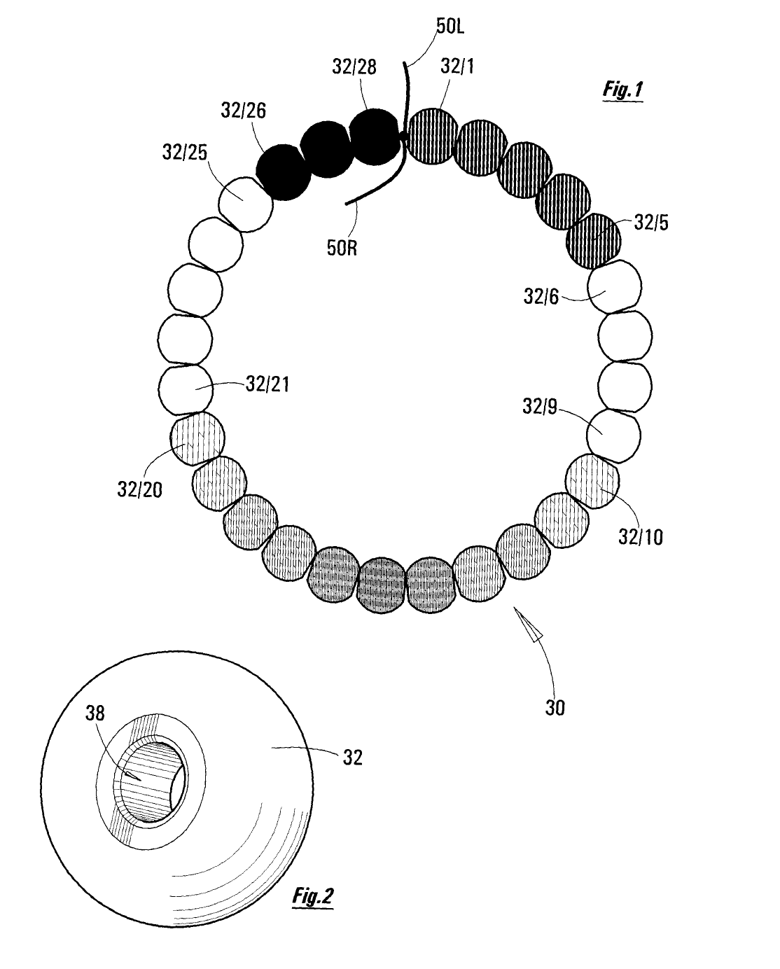

In the depicted bracelet, the twenty-eight beads 32 are simple wooden beads, one of which is shown in FIG. 2. Each bead is spherical. Each bead has a cylindrical through-hole 38. The wooden sphere is flattened around the two mouths of the through-hole 38, and the mouths are chamfered, as shown. Such beads are readily available as a purchasable product, or the beads can be made specially.

The beads 32 are identical as to shape and size. However, the beads are distinguished from each other by markings. In the example, the markings are the different colours of the beads.

In FIG. 1, the bead numbered 32/1 may be regarded as the start-bead of the cycle. The five beads numbered 32/1 to 32/5 are coloured red, or otherwise marked to indicate “period /menstruation/bleeding” days. The four beads numbered 32/6 to 32/9 are coloured white, or otherwise marked to indicate “neutral days”. The eleven beads numbered 32/10 to 32/20 are coloured pink, or otherwise marked to indicate “fertile days”. The five beads numbered 32/21 to 32/25 are coloured white, or otherwise marked to indicate “neutral days”. The three beads numbered 32/26 to 32/28 are coloured black or otherwise marked to indicate “PMS days” (PMS =premenstrual syndrome).

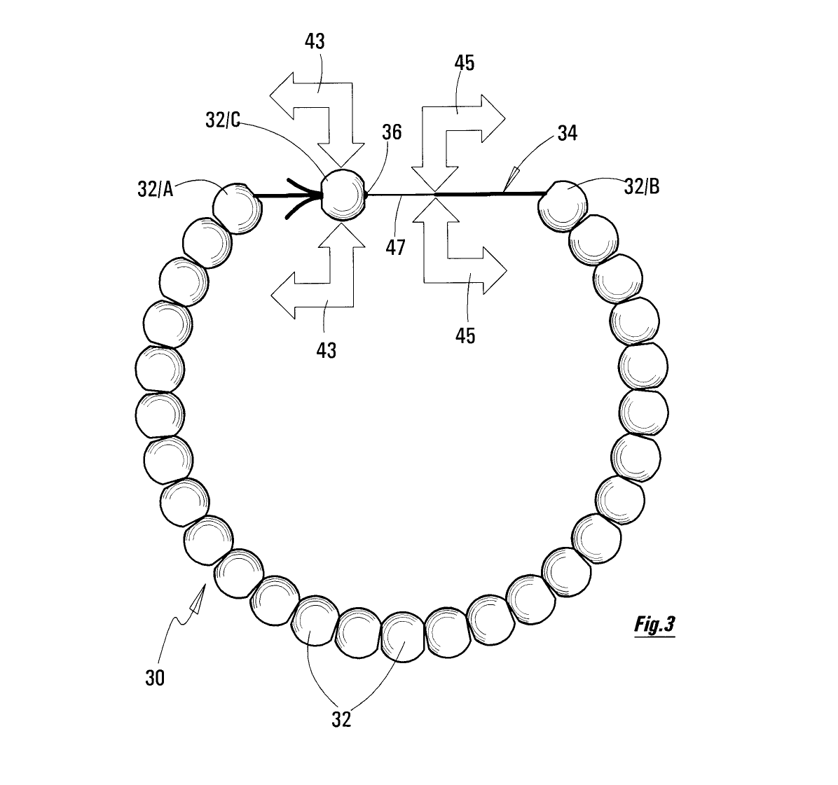

Preferably, the group of eleven beads 32/10 to 32/20, which can be coloured e.g pink to indicate “fertile days”, are graded as to the intensity of the colour. That is to say: the middle beads of the group are a deep pink, while the intensity of the colour has been faded in the beads towards the ends of the group of eleven. The end beads (32/10 and 32/20) of this group are almost white—i.e almost the same colour as the “neutral” beads. The deeper colour signifies the greater degree of potential fertility during those middle days. (In FIG. 3 and FIG. 8, the markings on (colours of) the beads have been omitted.)

The knot 36 serves as a day-marker of the menstrual-cycle monitoring device. The loose ends of the knot 36 protrude, as shown in FIG. 1, and are readily visible to the user, such that there can be no mistaking the location of the knot in relation to the beads.

The user establishes a daily routine of pulling the knot through one bead, i.e only one bead, each day, to mark the passage of the days. The structure of the bracelet is such that it is very easy to slip the bracelet off, to simplify the daily routine task of pulling the knot through the next bead.

In FIG. 3, the user is pulling the knot 36 through one of the beads. The cord 34 is of elastic material. That is to say, the cord will stretch when put under tension, and then will recover resiliently when the tension is released. When the bracelet 30 was being manufactured, the knot 36 was tied in such position as to make the loop of such length that the cord that makes up the loop is slightly stretched by the presence thereon of the twenty-eight beads 32. This condition is illustrated in FIG. 1.

The manner in which the knot 36 is pulled through the hole 38 in the bead 32 will now be described.

The cord 34 being elastic, the bracelet 30 can easily, by hand-manipulation, be arranged into the configuration shown in FIG. 3. In FIG. 3, the cord 34 has been stretched (i.e the circumference or hoop-length of the loop has been increased) whereby, now, the sum of the widths of all the beads 32 is smaller than the now-stretched hoop-length of the loop. Thus, the length of the now-stretched elastic cord between the beads 32/A and 32/B is exposed, as shown.

In FIG. 3, the user is pulling the knot 36 through the bead 32/C. Just before the condition shown in FIG. 3, the knot 36 was positioned between the beads 32/A and 32/C. The arrows 43 in FIG. 3 indicate that the bead 32/C is being gripped in the user's left hand, and is being urged in the leftwards direction. Similarly, the arrows 45 indicate that the cord 34 is being gripped in the user's right hand, and the cord is being urged rightwards. In FIG. 3, as shown, the knot 36 is just about to emerge out of the through-hole 38, on the right side of the bead 32/C.

The user pulls the knot 36 through the bead 32/C by pulling on the cord 34. Usually, to do this, the user holds the cord, in the location of the arrows 45, between the thumb and first finger of the right hand, while holding the bead 32/C between the thumb and first finger of the left hand.

The portion 47 of the cord 34, between the knot 36 and the user's right-hand, is under tension. In FIG. 3, the portion 47 being stretched, the diametral thickness of the portion 47 of the cord is reduced. This reduced thickness of the cord, in the stretched portion 47, is depicted in FIG. 3.

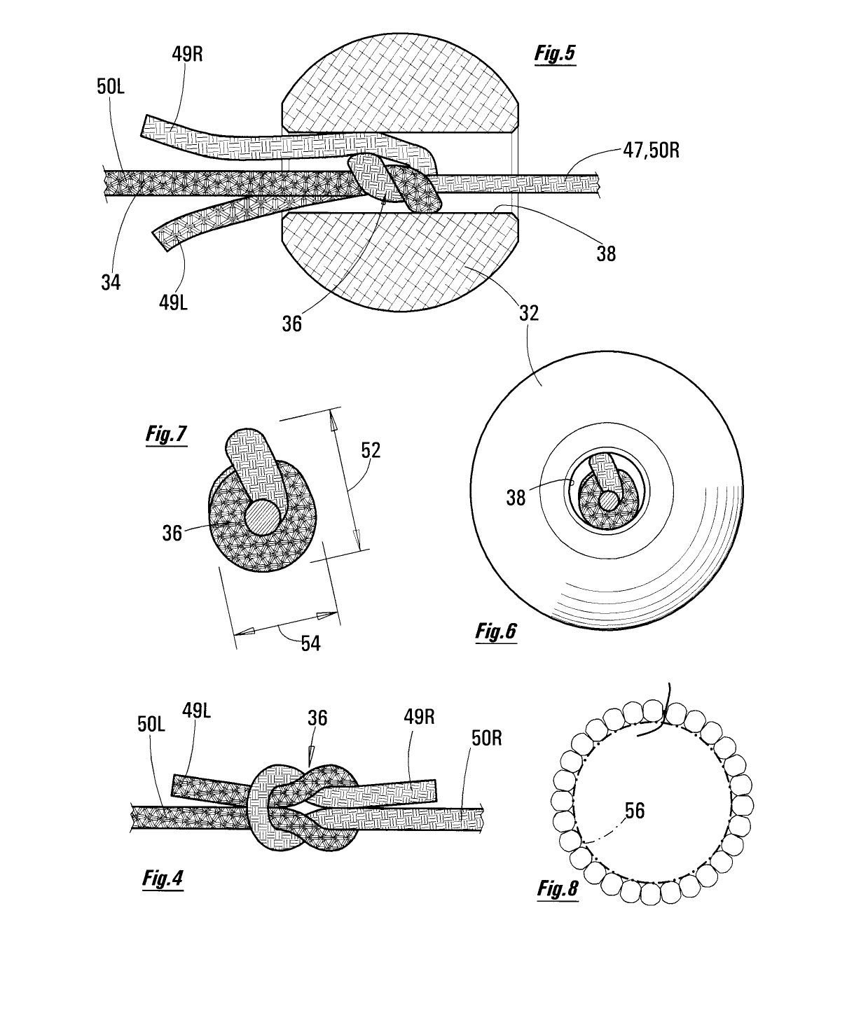

Preferably, the knot 36 is a reef-knot, as shown in FIG. 4. After the knot is tied, the portions of the cord that form the endless loop are referred to as the left and right working-ends 49L,49R of the knot. The other two portions of the knot are referred to as the left and right loose-ends 50L,50R of the knot. In FIG. 1, as can be observed, the day-by-day position of the knot is very clearly apparent, given the readily-noticeable loose-ends 50L,50R. The loose ends 50L,50R are not visually obtrusive, however, and do not detract from the appearance of the bracelet 30 as a decorative item.

FIG. 5 shows the situation inside the through-hole 38 of the bead 32, as the knot is being drawn in the rightwards direction through the hole.

The reef-knot is known, generally, as a reasonably secure and stable knot. However, the security of the reef-knot applies when the knot is used in the normal way, in which tension is applied between the two working-ends of the knot. In the present case, as shown in FIG. 5, the forces are applied to the knot in an unusual way. It will be understood that both of the loose-ends 49L,49R of the knot 36 are in a trailing relationship with respect to the knot 36 as the knot passes through the hole 38.

Thus, in FIG. 5, the right-side working-end 49R is under heavy tension; but that tension is reacted—not by the other working-end 49L, but—by the friction of the interaction between the outer-profile of the knot and the wall of the through-hole 38 in the bead. In fact, as can be seen, the right-side loose-end 50R protrudes from the knot to the left, and becomes one of the stressed areas of the knot.

This very unusual manner of stressing a reef-knot can be seen in FIG. 5. It has been found that the knot, when tied as a reef-knot, and when used in the manner as described and depicted, is in fact highly secure.

In fact, it is desired that the knot of the bracelet should remain always tight during the life of the bracelet. The knot should be highly resistant to coming loose—whether by working itself loose as a consequence of day-to-day operative usage of the bracelet, or as a result of inadvertent (or even deliberate) actions on the part of the user.

It might be the case that a reef-knot might not, in general, be secure against working loose, when stressed in the unusual manner shown in FIG. 5. However, it is recognized that the fact that the cord 34 is elastic, has an effect on the security of the knot 36.

When an elastic cord is pulled and stretched, and elongates elastically, its diametral thickness is reduced proportionally to the degree of stretching. Thus, when the knot 36 is pulled tight, the portions of the cord other than the loose-ends 50L,R are reduced in thickness. When the tension is then released, those portions of the cord that lie confined within the knot 36 cannot then, because of the tightness (and of the friction resulting from the tightness), expand and regain their natural (i.e unstressed) thickness.

As a result, once the knot 36 has been pulled tight, and the inner portions of the knot having been squeezed together, the knot 36 remains very tight. Thus, the fact of the cord 34 being elastic greatly increases the resistance of the knot 36 to working itself loose, especially once the knot has been pulled tight.

Elastic cord also usually has a higher coefficient of friction than non-elastic cord or thread, which also increases the knot's resistance to working loose.

FIG. 6 shows the outer cross-sectional profile of the knot 36, as it lies in the through-hole 38. FIG. 7 shows the knot outside the hole in the bead, and shows the maximum diameter 52 and the minimum diameter 54 of the profile. Again, it will be seen that the outer-profile of the knot includes the right-side loose-end 50R.

The size of the cord, the type of knot, and the size of the through-hole, are selected such that the outer cross-sectional profile of the knot is a tight fit in the (cylindrical) cross-sectional profile of the walls of the hole 38.

The knot 36 does not completely fill the whole cross-sectional area of the hole. The outer profile of the knot is uneven. That is to say (referring to FIG. 6), although protruding portions of the outer profile of the knot are pressing tightly against the walls of the hole 38, other portions (being recessed portions) of the outer profile of the knot make no contact at all with the walls of the hole 38. In the portions of the profile where there is heavy contact, there is correspondingly heavy friction between those portions of the outer profile of the knot that are in tight contact with the hole 38, and the walls of the hole.

What happens, as the knot 36 is drawn into, and passes through, the hole 38, is that the protruding areas of the outer profile of the knot are distorted inwards, until these areas can be, and are, contained within the right-cylindrical walls of the through-hole 38. The force required to distort the outer profile of the knot in this manner, and to overcome the friction of the movement of the squeezed knot through the hole in the bead, comes from the force supplied by the user pulling on the cord 34.

The designer of the bracelet seeks to create conditions in which the force required to pull the knot through the hole in the bead should not be so large as to damage the cord—bearing in mind that the knot is intended to be pulled through one of the beads, once a day, throughout the whole life of the bracelet. At the same time, the designer should see to it that the force required to pull the knot through the hole should not be so light that the knot can or might slip through the bead absent the deliberate intention of the user to make it do so.

The manner in which the knot 36 interacts with the through-hole 38 enables the knot to be able to distort in the advantageous manner as described, i.e enables the knot to undergo just the right amount of compression of the outer portions of the profile of the knot, whereby (within limits) the knot can accommodate itself to variations in the size of the hole in the bead. Thus, the knot is tolerant of variations in the hole diameter. The beads are not precision instruments, and the holes do vary.

The dimensional relationship between the elastic cord 34, the knot 36, and the through-hole 38 in the bead will now be described.

In the exemplary bracelet, the bead is a sphere measuring 8.13 millimetres diameter, overall. The flattened sides of the bead are 7.09 mm apart. The through-hole 38 is cylindrical, having a diameter of 2.41 mm. In the finished bracelet, the twenty-eight beads are under slight pressure, one against the other, due to the knotted loop of elastic cord 34 being under slight residual tension. The finished bracelet 30 being in its unstressed state, a circle inscribed inside the beads of the bracelet (being the circle 54 in FIG. 8) is six centimetres in diameter (=nineteen cm circumference).

As to the cord 34, the hoop-length of the loop in the finished bracelet is about twenty cm. At this size, the bracelet is a very comfortable fit on a girl's wrist, on a one-size-fits-(nearly)-all basis. The bracelet is not tight on the wrist, but is unlikely to fall off the user's hand accidentally; yet the elasticity of the cord means the bracelet can easily be slipped off, when the user wishes to do that. The ease of slipping the bracelet off can be important in making the daily task of moving the knot through the next bead simple and convenient.

The elastic material of the cord is such that the cord can be stretched to about double its unstretched dimensions, before reaching its elastic limit. Thus, the basic twenty-cm loop can be stretched to a hoop-length of about forty cm. The unstretched cord is of 1.20 mm diameter; stretched to its elastic limit, the diameter of the cord shrinks to 0.61 mm diameter.

It will be seen from FIGS. 5,6,7 that the knot 36 provides an aggregate of four thicknesses of the cord, when the knot is passing through the hole 38 in the bead. These four thicknesses are slightly displaced, in the axial direction of the hole 38, from each other. Thus, it will be seen and understood how readily the knot can accommodate its four thicknesses of the cord to the diameter of the hole, even though the diameter of the holes can vary, bead to bead. Thus, the force needed to pull the knot through the hole is reasonably constant, bead to bead, and bracelet to bracelet, despite significant manufacturing variations, especially of the bead.

When being pulled through the hole 38, as in FIG. 5, the knot 36 does not tend to become jammed in the hole 38. In fact, as mentioned, the size of the hole 38 can vary, within a wide tolerance, and yet still the force required to pull the knot through the hole stays reasonably constant.

Incidentally, by contrast, if, when the knot 36 is partway through the bead, the user were to change her mind, and were now to start to pull the knot back out of the bead (i.e to pull the knot leftwards, in FIG. 5), it is likely, then, that the knot would jam in the hole. When tension is applied to the left-side working-end 49L of the knot, after the knot has already been pulled into the hole, the four thicknesses of the cord now tend to ride up onto each other, and to interfere with each other.

Again, when tension was applied to the right-side working-end 49R, the four thicknesses arranged themselves slightly one behind the other, as will be understood from FIG. 5. But when tension is applied to the left-side working-end 49L, the knot cannot contract in overall size in order to accommodate itself to the size of the hole; rather, the knot tends to increase in size as a result of leftwards-applied tension, and the four thicknesses pile up on each other, whereby the knot very likely jams in the hole. By contrast, when tension is applied to the right-side end 49R, then the four thicknesses align one behind the other, and the knot can squeeze itself progressively into the hole.

It will be understood that this jamming of the knot under leftwards tension only occurs if the knot is already partway into the hole when the leftwards tension is applied. If the leftwards tension were to be applied while the loose ends 50L,50R both lie outside the bead, of course the knot could be pulled leftwards through the bead—as a mirror image of FIG. 5.

The tendency of the knot 36 to jam when pulled in reverse is not significant, from the standpoint of the daily use of the bracelet. The tendency was mentioned in order to highlight the contrast with the intended manner of pull-through, as shown in FIG. 5, in which the knot automatically aligns itself into the highly favourable configuration shown in FIGS. 5,6.

In order for the knot to serve as the day-marker, in the convenient manner as described herein, the cord, the knot, and the hole in the bead, must complement each other, in ways that will now be described.

The cord 34 should have a good degree of elasticity. Elastic cord is cord that can be stretched resiliently up to its elastic limit. Beyond its elastic limit, further tension applied then to the cord starts to cause damage to the cord, in that the cord takes on a permanent set, and does not revert to its unstretched length when released. Further tension can result in breakage of the cord, of course.

For present purposes, when a ten cm length of unstretched cord is stretched to a length of sixteen cm, that cord is said to have been stretched by sixty percent. The elastic stretch limit of the cord may be expressed as the percentage stretch at which the cord reaches its limit of elasticity.

In order for the bracelet 30 to be able to function in the manner described herein, the cord 34 should have an elastic limit of not less than fifty percent. In the exemplary bracelet described herein, the elastic cord had an elastic limit of about a hundred percent. That is to say: a twenty cm length of the cord can be stretched to forty cm.

Now, when the knot is actually being pulled through the hole in the bead, the portion 47 of the elastic cord, now stretched, should be stretched at a level that is well below its elastic limit. Thus, suppose, in a particular case, that the elastic limit of a particular cord is ninety percent, in that case the designers should aim for the portion 47 of the cord, during pull-through (FIGS. 3,5), to be stretched to about half of its elastic limit, i.e the cord should be stretched by about forty-five percent during pull-through.

That is to say: designers should aim to provide a combination of the elastic cord 34, the knot 36, and the size of the hole 38 in the bead, such that the magnitude of the pull-through stretch is about half of the elastic limit of the cord. If the pull-through stretch is too high, the possibility arises of the cord being damaged by overstretching, during the life of the bracelet. Designers should aim for the pull-through stretch (in the portion 47 of the cord) to be no more than a maximum of seventy percent of the elastic-limit.

This is equivalent to stating that the designers should see to it that the combination of cord+knot+hole is such that the force required to pull the knot through the bead is no more than seventy percent of the force required to stretch the cord to its elastic limit.

On the other hand, the pull-through stretch should not be so light that the user might inadvertently pull the knot through the bead-hole, e.g when running the bracelet through her fingers while absently playing with it, for example. From that standpoint, the designers should see to it that the combination of cord+knot+hole should give rise to a pull-through stretch of the cord of not less than twenty percent of the elastic limit of the cord.

Again, this is equivalent to stating that the designers should see to it that the combination of cord+knot+hole is such that the force required to pull the knot through the bead is no less than twenty percent of the force required to stretch the cord to its elastic limit.

Again, the knot 36 is a tight fit in the through-hole 38 of the bead 32. That is to say, the user can pull the knot 36 through the bead, but significant force must be applied to the cord in order to do this. The intent is that the knot cannot pass through the hole accidentally or inadvertently, but that, when deliberate force is indeed applied to the cord, the knot can be pulled through by simple hand manipulation of the cord and the beads.

Some of the key features that define the new technology will now be described.

The menstrual-cycle monitoring device includes an endless loop of cord, and includes a set of beads, the beads being formed with respective through-holes. The beads are threaded onto the cord, the cord passing through the holes in the beads.

The set of beads is configured and arranged, one after another, in a ring, in such manner that the beads represent the ongoing days of the menstrual cycle. The beads are provided with respective markings, indicative of days in the sectors of the cycle. The markings differentiate at least between the menstruation sector of the cycle, and the peak-fertility sector.

The cord is provided with a local swelling or promontory, which is a fixture on the cord. The promontory is small enough that the promontory can be pulled through the hole of one of the beads, by a person manually pulling the cord through the bead. The promontory is of such size and shape, in relation to the through-hole in the bead, that the promontory can be drawn through the hole, but only by distorting the promontory.

The device includes a day-marker, of such structure as to provide a visible indication of the location of the promontory, in relation to the ring of beads.

In the bracelet as depicted and described, the promontory is provided by the knot, and the day-marker is provided by the loose-ends of the knot.

Preferably, the cord is elastic, and the knot is a reef-knot. The combination of knot+cord+hole should be such that the force required to pull the knot through the bead is between twenty percent and seventy percent of the force required to stretch the cord to its elastic limit.

The device is primarily intended as a learning tool. The device is especially aimed at pubescent and adolescent girls, and especially at girls whose schools, for some reason, teach them nothing about the developing sexual and reproductive aspects of their bodies. The device is intended to educate the girls about their menstrual cycle, in a most memorable way. The device is intended to educate users as to the actuality of the sectors of the menstrual cycle, and as to their importance in the fertility cycle. The device is intended to teach the concept that the menstrual cycle is indeed cyclic, repeating over and over.

The device is intended to make it easy for the girl user to determine when/whether her periods have settled down, as to their length/frequency/regularity.

In the case of a girl whose body has settled into a cycle that differs substantially from the usual twenty-eight day cycle, the device is intended to alert the girl to the fact that her frequency does differ from the usual. Of course, it is not unusual for girls' menstrual parameters to vary—especially during adolescence, but also in adulthood—and the device is useful for highlighting that that is happening.

The device does not require the user to have a cycle that is always and only twenty-eight days long. In the case of a girl or woman whose cycle settles e.g to twenty-seven days, she can simply advance the marker on the first day of her period, and little is lost by way of accuracy of the menstrual status indicated by the beads.

Similarly, if her cycle should settle to twenty-nine days, she can simply refrain from moving the marker onto the first of the “period days” until her period actually starts. And the girl or woman, even if she has lost touch with just where she is in her cycle, can hardly make a mistake when it comes to resetting the device to the first red bead on the first day of her period. Thus, there is no need for the number of beads to be tailored to the particular cycle of the user.

Naturally, there will be some occasions when a user might forget to advance the marker that day, or might forget that she has already advanced the marker, and advance it twice that day. If she should lose track, she can readily reset the device on day-one of her next period.

As mentioned, a major aim of the device is to educate a young girl to the concept, and the fact, of her regular repeating ongoing monthly cycle, and that aim can be realised even though she might be (slightly) forgetful as to daily operation.

The user might wish to use the device as an aid to conceiving. In such case, she had better be well-disciplined as to the need to attend to the day-by-day operation of moving the marker. Reliance should not be placed on the device for contraceptive purposes.

The device may be worn all the time, as a decorative bracelet. However, the educational function of the device is still present even if the device is e.g carried in a purse. The device ideally needs to be operated daily, of course; much of its educational impact might be lost if the user leaves it for a day or two and then tries to catch up.

The numerals that appear in the accompanying drawings are summarized as:
30 bracelet
32 beads (spherical, wooden)
34 cord (elastic)
36 knot in cord
38 through-hole in bead
43 arrows indicating forces exerted by left hand
45 arrows indicating forces exerted by right hand
47 stretched portion of cord
49L,R loose-ends of knot
50L,R working-ends of knot
52 maximum diameter of outer profile
54 minimum diameter of outer profile
56 inscribed circle.

1. A menstrual-cycle monitoring device, characterized by the following combination of features:
the device includes an endless loop of cord;
the device includes a set of beads, the beads being formed with respective through-holes;
the beads are threaded serially onto the cord, the cord passing through the holes in the beads;
the set of beads is configured and arranged, one after another, in a ring, in such manner that the beads represent the ongoing days of the menstrual cycle;
the beads are provided with respective markings, indicative of days in the sectors of the cycle;
the markings differentiate at least between the menstruation sector of the cycle, and the peak-fertility sector;
the cord is provided with a local swelling or promontory, which is a fixture on the cord;
the promontory is small enough that the promontory can be pulled through the hole of one of the beads, by a person manually pulling the cord through the bead;
the promontory is of such size and shape, in relation to the through-hole in the bead, that the promontory can be drawn through the hole, but only by distorting the promontory; and
the device includes a day-marker, of such structure as to provide a visible indication of the location of the promontory, in the ring of beads.

2. As in claim 1, wherein:
the endless loop of cord is formed from a length of cord having two ends;
the two ends of the cord have been tied together in a knot, to form the loop;
the knot is the promontory;
the configurations of the loop and the knot are such that two working-ends of the loop are knotted together at the knot, and the two ends of the cord form two loose-ends of the knot;
at least one of the loose-ends protrudes outwards from the knot; and
the protruding loose-end is the day-marker.

3. As in claim 2, wherein:
the knot is larger than the through-hole;
whereby the knot has to be reduced in size in order for the knot to be pulled through the hole in the bead.

4. As in claim 1, wherein:
the cord is elastic, in that when the cord is pulled in tension the cord stretches, and in that when the tension is released the cord reverts resiliently to its unstretched length;
the cord has an elastic-limit, in that if the cord were to be stretched beyond its elastic-limit, further tension applied then to the cord would damage the cord;
the elastic-limit of the cord is at least fifty percent.

5. As in claim 2, wherein:
the structure of the device is such that, upon a person applying pull-through tension to the cord, the knot enters the hole in the bead;
the pull-through tension in the cord is the force that is applied to the cord and results in the knot passing through the hole in the bead, and the pull-through stretch is the stretch of the cord when subjected to the pull-through tension;
the size and elasticity of the cord, the form and configuration of the knot, and the size of the hole in the bead, are related to each other in such manner that the pull-through stretch is no more than seventy percent of the elastic-limit of the cord.

6. As in claim 5, wherein the size and elasticity of the cord, the form and configuration of the knot, and the size of the hole in the bead, are related to each other in such manner that the pull-through stretch is no less than twenty percent of the elastic-limit of the cord.

7. As in claim 1, wherein:
the beads are twenty-eight in number;
the device is sized and structured as a bracelet;
an inscribing circle of the bracelet is between five and seven cm in diameter.

8. As in claim 2, wherein:
the knot has an outer profile, which is of variable diameter;
a diameter of the outer profile is defined as a line having the following characteristics:
(a) the diameter joins two oppositely-facing points on opposite sides of the outer profile of the knot;
(b) in respect of a line drawn through either end of the diameter, at right angles to the diameter, no portion of the profile protrudes outside that line;
(c) the length of the diameter is measured when the knot lies outside the holes in the beads;
the maximum diameter of the outer profile of the knot is so large that, even after the knot has been pulled tight, to the elastic limit of the cord, still the maximum diameter of the profile of the knot is larger than the diameter of the hole, whereby the knot cannot be pulled through the hole in the bead without being distorted;
the minimum diameter of the outer profile of the knot is so small that, even after the knot has been distorted and squeezed into the hole in the bead, still there is some daylight between the wall of the hole and the distorted outer profile of the knot.

9. As in claim 1, wherein:
the ring of beads includes at least four contiguous beads that are of colour R;
the left-end bead of these R beads is termed the first bead of the ring of beads;
the next adjacent bead to the right of the first bead is termed the second bead, and the rest of the beads are numbered consecutively around the ring of beads;
at least the nine beads that constitute the eleventh bead through the nineteenth bead are of colour P;
at least the two beads immediately to the left of the first bead are of colour B;
the colours R, P, B, are distinctive and distinguishable from each other.

10. As in claim 9, wherein:
the beads are twenty-eight in number;
the first through fifth beads are coloured red;
the sixth through ninth beads are coloured white;
the tenth through twentieth beads are coloured pink;
the fifteenth bead is coloured a deeper pink than the tenth and twentieth beads;
the twenty-first through twenty-fifth beads are coloured white; and
the twenty-sixth through twenty-eighth beads are coloured black.
